(19) United States
(12) Patent Application Publication
(10) Pub. No.: US 2019/0070232 A1
(43) Pub. Date: Mar. 7, 2019

(54) METHODS OF CLONING PROPHAGES AND PRODUCING LYTIC PHAGE PARTICLES

(71) Applicant: MASSACHUSETTS INSTITUTE OF TECHNOLOGY, Cambridge, MA (US)

(72) Inventors: Timothy Kuan-Ta LU, Cambridge, MA (US); Robert James CITORIK, Kingston, NH (US)

(73) Assignee: MASSACHUSETTS INSTITUTE OF TECHNOLOGY, Cambridge, MA (US)

(21) Appl. No.: 16/080,468

(22) PCT Filed: Mar. 5, 2018
(86) PCT No.: PCT/US2018/020848

§ 371 Date: Aug. 28, 2018

Related U.S. Application Data
(60) Provisional application No. 62/467,501, filed on Mar. 6, 2017.

(51) Int. Cl. A61K 35/76; C12N 15/73; C12N 15/74; C12N 15/10; A61P 31/12

(52) U.S. Cl. CPC A61K 35/76; C12N 15/73; A61P 31/12; C12N 15/1096; C12N 15/74

(57) ABSTRACT

Disclosed herein are novel methodologies for cloning prophage genome sequences that are identified from target organisms or DNA sequencing data and that contain mutations that decrease the function of prophage repressor proteins and for producing lytic phage particles with decreased prophage repressor protein function.

### RELATED APPLICATIONS

This application is a national stage filing under 35 U.S.C. 371 of International Patent Application Ser. No. PCT/US2018/020848, filed Mar. 5, 2018, which claims priority under 35 U.S.C. § 119(e) to U.S. provisional application No. 62/467,501, filed Mar. 6, 2017, the contents of each of which is incorporated herein by reference in their entirety.

### GOVERNMENT SUPPORT

The invention was made with government support under Grant No. HDTRA1-14-1-0007 awarded by the Defense Threat Reduction Agency. The government has certain rights in the invention.

### FIELD

Disclosed herein are novel methodologies for cloning prophage genome sequences that are identified from target organisms or DNA sequencing data and that contain one or more mutations that decrease the function of prophage repressor proteins and novel methodologies for producing lytic phage particles with decreased prophage repressor protein function.

### BACKGROUND

Temperate bacteriophages possess both lytic and lysogenic cycles. In the lytic cycle, the phage replicates and lyses the host bacterial cell, and in the lysogenic cycle, phage DNA enters a relatively silenced prophage state, which often includes incorporation into the host bacterial genome (FIG. 1). The phage cycle is controlled by multiple factors, the most dominant of which is the presence of the major repressor protein, which functions as a genetic switch. Various phage repressor proteins have been identified (e.g., Hammer J. A., et. al., Viruses, E213 (2016)).

### SUMMARY

Phage engineering using the techniques of molecular biology has found wide application, including the stimulation of bacterial cell death. For example, bacteriophages have been engineered to express antimicrobial peptides (AMPs) and factors that disrupt intracellular processes, leading to rapid, bacterial death (e.g., Krom R. J., et al., Nano. Lett., 15(7): 4808-13 (2015); Bikard D., et. al., Nat. Biotechnol., 32(11): 1146-50 (2014); Citorik R. J., et. al., Nat. Biotechnol., 32(11): 1141-45 (2014); Westwater C., et. al., Antimicrob. Agents Chemother., 47(4): 1301-7 (2003); Hagens S. and Bläsi U., Lett. Appl. Microbiol., 37(4): 318-23 (2003)). Alternatively, lytic death pathways have been manipulated through the engineering of bacteriophages that express lytic enzymes or peptides (e.g., WO 2016/100389, US 2016/0186147, US 2015/0050717, US 2014/0161772, US 2012/0244126, US 2012/0134972, WO 2010/141135, WO 2010/136754, US 2009/0155215, CN 101067123). However, to date, the engineering of prophages as lytic phages through the targeted, intentional mutation or deletion of phage repressor proteins or of DNA sequences bound by phage repressor proteins has remained unexplored.

Disclosed herein are novel methodologies for cloning prophage genome sequences that are identified from target organisms or DNA sequencing data and that contain one or more mutations that decrease the function of prophage repressor proteins and novel methodologies for producing lytic phage particles with decreased prophage repressor protein function.

In one aspect, methods of cloning a prophage are provided. The methods include: obtaining a prophage genome sequence, mutating the prophage genome sequence in a sequence of the genome that decreases the function of a repressor protein, related protein, or regulatory region thereof, and assembling the mutated prophage genome by either yeast assembly or in vitro assembly, optionally wherein the phage genome is isolated. In some embodiments, the prophage genome sequence is obtained from a phage-host cell.

In some embodiments, the prophage genome is obtained by PCR, de novo synthesis, or digestion of cellular DNA. In some embodiments, the mutated prophage genome sequence comprises at least one mutation in the sequence encoding for a protein that regulates the lysogenic cycle. In some embodiments, the mutated prophage genome sequence comprises at least one mutation in the sequence encoding for the phage repressor, wherein the mutation decreases the function of the repressor. In some embodiments, the mutated prophage genome sequence comprises one or more deletions, insertions and/or substitution mutations. In some embodiments, the mutated prophage genome sequence comprises a knockout (e.g., complete deletion) of the phage repressor gene. In some embodiments, the mutation is in the DNA-binding domain of the repressor, or in a region that reduces stability of the protein.

In some embodiments, the mutated prophage genome sequence comprises at least one mutation in a sequence that participates in regulating the lysogenic cycle. For example, in some embodiments, the mutated prophage genome sequence comprises at least one mutation in a sequence encoding at least one binding site of and/or the promoter sequence of the phage repressor.

In some embodiments, the mutated prophage genome is further modified such that it encodes a phage that obligately kills its host cell. This can be achieved by addition of a constitutive toxic function to the mutated prophage genome, such as a sequence encoding a constitutively expressed toxic molecule (e.g., one or more prokaryotic toxins, antimicrobial peptides, and/or nucleases).

In another aspect, methods of producing lytic phage particles are provided. The methods include: assembling a mutated prophage genome and introducing the mutated prophage genome into a host-cell or into an in vitro cell-free extract.

In some embodiments, the cell-free extract is generated from a bacterial strain. Any bacterial strain can be used that executes functions of the mutated prophage genome required for producing phage particles that include the mutated prophage genome. In some embodiments, the cell-free extract is generated from the target strain of the lytic phage. In some embodiments, the phage particles are engineered entirely in vitro.

These and other aspects are described in more detail below.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1. Schematic overview of the lytic and lysogenic phage cycles. In the lytic cycle, the phage replicates and lyses the host cell, and in the lysogenic cycle, phage DNA is incorporated into the host genome (prophage). The phage cycle is controlled by the presence and abundance of active repressor protein, along with secondary factors.

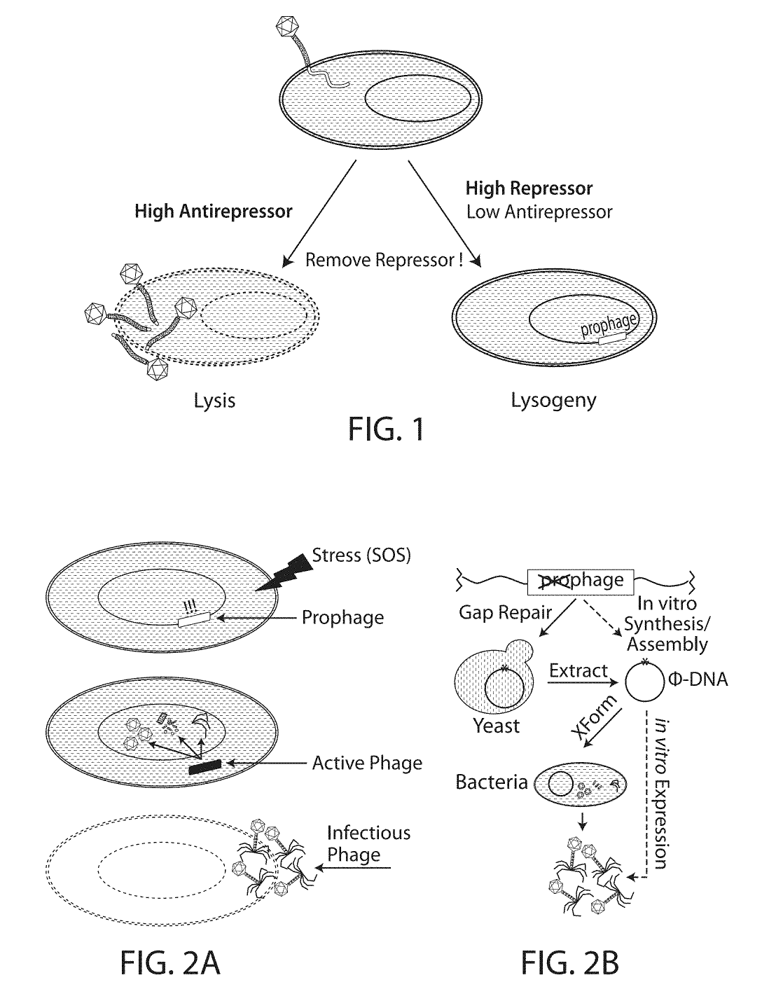

FIGS. 2A-2B. Cloning and booting mutant prophages from target host strains. FIG. 2A. Schematic overview of prophage activation and the generation of infectious phage particles. FIG. 2B. Schematic overview of the synthesis or cloning of prophage genome sequences that contain mutations that decrease the function of a prophage repressor protein and the production of lytic phage particles with decreased prophage repressor protein function. The starting prophage genome may be obtained through various methods including extraction from bacteria or phage particles or de novo synthesis.

FIG. 3. Cloning and rebooting E. coli phage N15. Schematic overview of the cloning of E. coli phage N15 genome sequences that contain mutations that decrease the function of a prophage repressor protein and the production of lytic phage particles with decreased prophage repressor protein function. Cloning was performed via both yeast-based assembly and in vitro digestion and ligation. The starting prophage genome may be obtained through various methods including extraction from bacteria or phage particles or de novo synthesis.

FIG. 4. Transmission electron microscopy of PEG-purified E. coli phage N15.

FIG. 5. Wild-type and forced lytic phage N15. Phage N15 repressor mutants constructed via both in vitro digestion and ligation (second row) or yeast-based assembly (third row) produced phages that yielded clear plaques relative to their wild-type controls (first and fourth rows, respectively).

FIGS. 6A-6D. Identification of prophages from K. pneumoniae (KPNIH31). FIG. 6A. Overview of the KPNIH strains used in this study, including their high-level CPS type and their susceptibility to the prophage from KPNIH31. FIG. 6B. Assays of KPNIH strain growth in the presence of various antibiotics. Strain KPNIH31 sustained growth in typical working concentrations of each antibiotic assayed. FIG. 6C. Schematic overview of the genetic composition of strain KPN1H31 (modified from Conlan et al., Sci. Transl. Med. (2014)). FIG. 6D. Genome data of strain KPNIH31 identifying a region containing a possible phage via PHAST (Zhou et al., Nucleic Acids Res. (2011)).

FIGS. 7A-7B. Rebooting of temperate phage preliminarily named ΦKpn852, derived from K. pneumoniae KPNIH31, from purified genome via E. coli transformation. FIG. 7A. Overview of the purification of ΦKpn852 genome and transformation into ELITE 10G cells. Transformed E. coli cells produced functional ΦKpn852 progeny that could be detected by spotting onto a double-agar lawn of K. pneumoniae KPNIH31 (natural E. coli phage N15 shown for comparison). Because E. coli can produce functional ΦKpn852, the pipelines from either FIG. 2B or FIG. 3 can be applied to reprogram the temperate phage into a lytic phage. FIG. 7B. Transmission electron microscopy of PEG-purified ΦKpn852.

### DETAILED DESCRIPTION

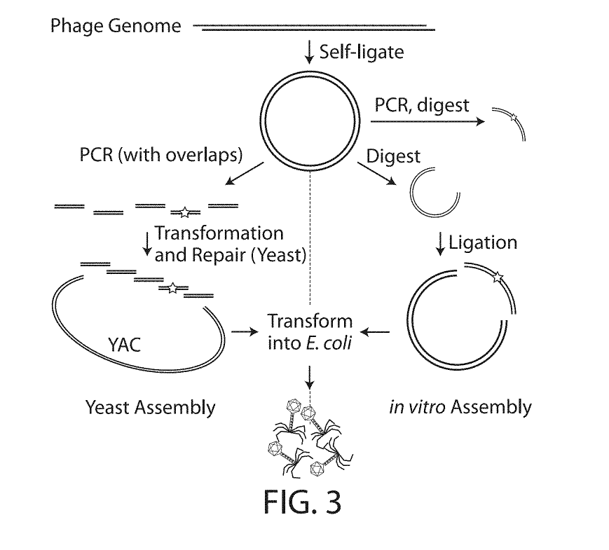

Disclosed herein are novel methodologies for cloning prophage genome sequences that are identified from target organisms or DNA sequencing data and that contain mutations that decrease the function of prophage repressor proteins and novel methodologies for producing lytic phage particles with decreased prophage repressor protein function (FIG. 2A-2B). In some embodiments, the methods of cloning a prophage disclosed herein include: obtaining a prophage genome sequence, mutating the prophage genome sequence in a sequence of the genome that decreases the function of a repressor protein, and assembling the mutated prophage genome by either yeast assembly or in vitro assembly, optionally wherein the phage genome is isolated.

As used herein, the term “phage” refers to both bacteriophages (i.e., bacterial viruses) and archaeophages (i.e., archaeal viruses), but in certain instances, as indicated by the context, phage may also be used as shorthand to refer specifically to a bacteriophage or archaeophage. Bacteriophage and archaeophage are obligate intracellular parasites that multiply inside a host cell by making use of some or all of the cell's biosynthetic machinery.

In some embodiments a phage is a member of an order selected from Caudovirales, Microviridae, Corticoviridae, Tectiviridae, Leviviridae, Cystoviridae, Inoviridae, Lipothrixviridae, Rudiviridae, Plasmaviridae, and Fuselloviridae. In some embodiments the phage is a member of the order Caudovirales and is a member of a family selected from Myoviridae, Siphoviridae, and Podoviridae.

“Bacterial virus” or “bacteriophage” refers to a virus that infects bacteria. In some embodiments the bacteria is a member of a phyla selected from Actinobacteria, Aquificae, Armatimonadetes, Bacteroidetes, Caldiserica, Chlamydiae, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Nitrospirae, Planctomycetes, Proteobacteria, Spirochaetes, Synergistets, Tenericutes, Thermodesulfobacteria, and Thermotogae. In some embodiments the phage is able to infect at least one Firmicutes selected from Bacillus, Listeria, Staphylococcus, and Clostridium. In some embodiments the phage is able to infect a member of Bacteroides. In some embodiments the phage is able to infect at least one Proteobacteria selected from Acidobacillus, Aeromonas, Burkholderia, Neisseria, Shewanella, Citrobacter, Enterobacter, Erwinia, Escherichia, Klebsiella, Kluyvera, Morganella, Salmonella, Shigella, Yersinia, Coxiella, Rickettsia, Legionella, Avibacterium, Haemophilus, Pasteurella, Acinetobacter, Moraxella, Pseudomonas, Vibrio, and Xanthomonas. In some embodiments the phage is able to infect at least one Tenericutes selected from Mycoplasma, Spiroplasma, and Ureaplasma.

“Archaeal virus” or “archaeophage” refers to a virus that infects archaea. In some embodiments the archaea is a Euryarcheota. In some embodiments the archaea is a Crenarcheota.

As used herein, “phage-host cell” or “host cell” refers to a cell that can be infected by a phage.

The term “obtaining” as used herein, relates to identifying and isolating a phage genome sequence. In some embodiments, the prophage genome sequence is identified from a phage-host cell. In some embodiments, the prophage genome sequence is identified from genome sequencing data. In some embodiments, the prophage genome is isolated by PCR, de novo synthesis, purification from functional phage particles, or digestion of cellular DNA. In some embodiments a phage genome comprises at least 1 kilobase (kb), at least 5 kb, at least 10 kb, at least 15 kb, at least 20 kb, at least 25 kb, at least 30 kb, at least 35 kb, at least 40 kb, at least 45 kb, at least 50 kb, at least 55 kb, at least 60 kb, at least 65 kb, at least 70 kb, at least 75 kb, at least 80 kb, at least 85 kb, at least 90 kb, at least 95 kb, at least 100 kb, at least 105 kb, at least 110 kb, at least 115 kb, at least 120 kb, at least 125 kb, at least 130 kb, at least 135 kb, at least 140 kb, at least 145 kb, at least 150 kb, at least 175 kb, at least 200 kb, at least 225 kb, at least 250 kb, at least 275 kb, at least 300 kb, at least 325 kb, at least 350 kb, at least 325 kb, at least 350 kb, at least 375 kb, at least 400 kb, at least 425 kb, at least 450 kb, at least 475 kb, at least 500 kb, or more.

As used herein, “repressor protein” refers to a transcriptional repressor that allows a phage to establish and maintain latency. Multiple prophage repressor proteins have been identified, including prophage repressors c1 and cB (e.g., Hammer J. A., et. al., Viruses, E213 (2016)).

As used herein, “mutation” may refer to a point mutation, an insertion, a deletion, a frameshift, or a missense mutation, and particularly a mutation that decreases function of the repressor. As used herein, “decreases function” refers to a decrease of at least 10%, 20%, 25%, 30%, 40%, 50%, 60%, 70%, 75%, 80%, 90% or up to 100% in the levels of repression generated by a prophage repressor protein. One skilled in the art can readily determine the repressive potential of prophage repressors via evaluation of gene expression, amount of cell growth or lysis, ability to form lytic phage particles, or otherwise.

In some embodiments, the mutated prophage genome sequence comprises a knockout of the phage repressor gene, such as a partial or complete deletion of the phage repressor gene. In other embodiments, the mutated prophage genome sequence comprises at least one mutation in the sequence encoding for the phage repressor, wherein the mutation decreases the function of the repressor. In some embodiments, the mutated prophage genome sequence comprises at least one mutation in a sequence encoding at least one binding site of the phage repressor. In some embodiments, the mutated prophage genome sequence comprises at least one mutation in a regulatory sequence involved in lysogeny.

Molecular techniques for yeast-based and Gibson assembly of DNA constructs are known in the art (e.g., US 2013/0122549). Additional recombination-based approaches are also known to those skilled in the art, including, but not limited to SLiCE. Moreover, it is anticipated that alternative genome editing techniques can be utilized in generating mutations of a prophage genome sequence including, but not limited to, zinc finger nucleases, transcription activator-like effector nucleases (TALENs), meganucleases, and CRISPR nuclease systems (e.g., Kiro R., et. al., RNA Biol., 42-4 (2014)).

In some embodiments, the mutated prophage genome is further modified such that it encodes a phage that obligately kills its host cell. This can be achieved by addition of a constitutive toxic function to the mutated prophage genome, such as a sequence encoding a constitutively expressed toxic molecule (e.g., one or more prokaryotic toxins, antimicrobial peptides, and/or nucleases). Such a modification to the mutated prophage genome to constitutively express a toxic function in the host cell would effectively prevent lysogenization by forcing lysis of the host cell.

In other embodiments, methods of producing lytic phage particles are disclosed herein that include: assembling a mutated prophage genome, and introducing the mutated prophage genome into a host cell or into an in vitro cell-free extract. In some embodiments, the mutated prophage genome is assembled by cloning a prophage from a cell comprising the steps of: obtaining a prophage genome sequence mutating the prophage genome sequence in a sequence of the genome that decreases the function of a repressor protein, and assembling the mutated prophage genome by either yeast assembly or in vitro assembly. In some embodiments, the phage genome is isolated.

Methods of producing phage particles via utilization of bacterial transformation or via in vitro assembly using a cell-free extract are known in the art (e.g., US 2013/0122549; Shin J., et. al, ACS Synth. Biol., 408-13 (2012)). In some embodiments, the cell-free extract is generated from a bacterial strain. In some embodiments, the cell-free extract is generated from the target strain of the lytic phage, or a related strain capable of producing functional phage. In some embodiments, the phage particles are engineered entirely in vitro.

### EXAMPLES

### Example 1

### Cloning of Mutant E. coli Phage N15 and Phage Particle Production

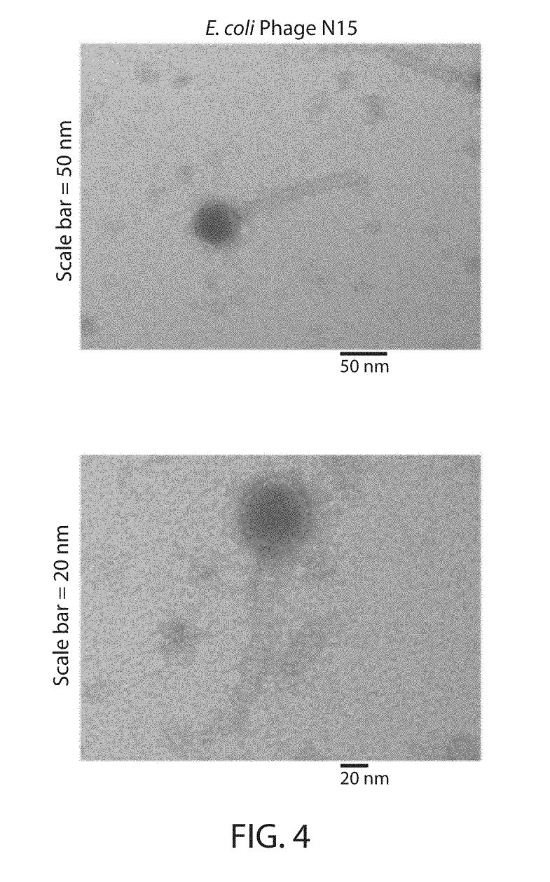

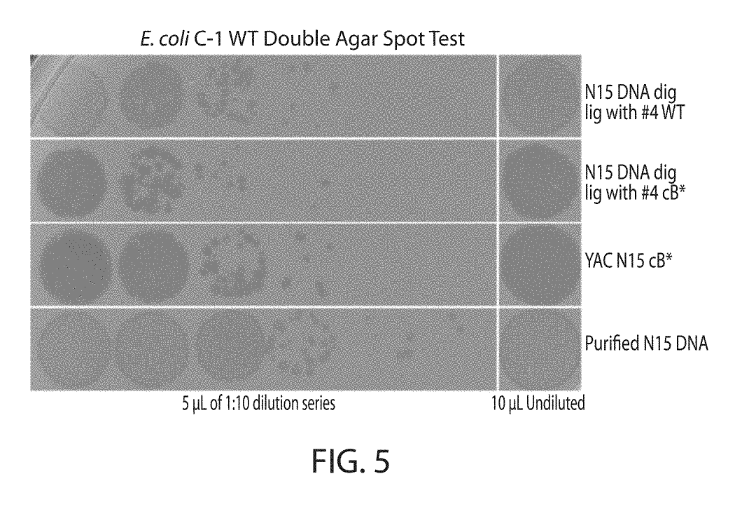

E. coli phage N15 genome sequences that contain either a wild-type protein sequence or a repressor null mutant protein sequence (through introduction of a premature stop codon) were cloned via both yeast-based assembly and in vitro digestion and ligation (FIG. 3). Cloned DNA was transformed into E. coli 10G ELITE Electrocompetent (Lucigen) cells and recovered in Lucigen recovery medium for at least 3 h. Crude wild-type and mutant bacteriophage samples were harvested by centrifugation and 0.2 μm filtration. PEG-8,000 was added to 10% w/v to precipitate phage particles, which were then concentrated through centrifugation and resuspension in SM buffer (FIG. 3). Transmission electron microscopy (TEM) was used to visualize samples prepared on Carbon Formvar grids and stained with uranyl acetate (FIG. 4). Phage genomic DNA was purified from PEG-concentrated phages using the Zymo Viral Purification kit.

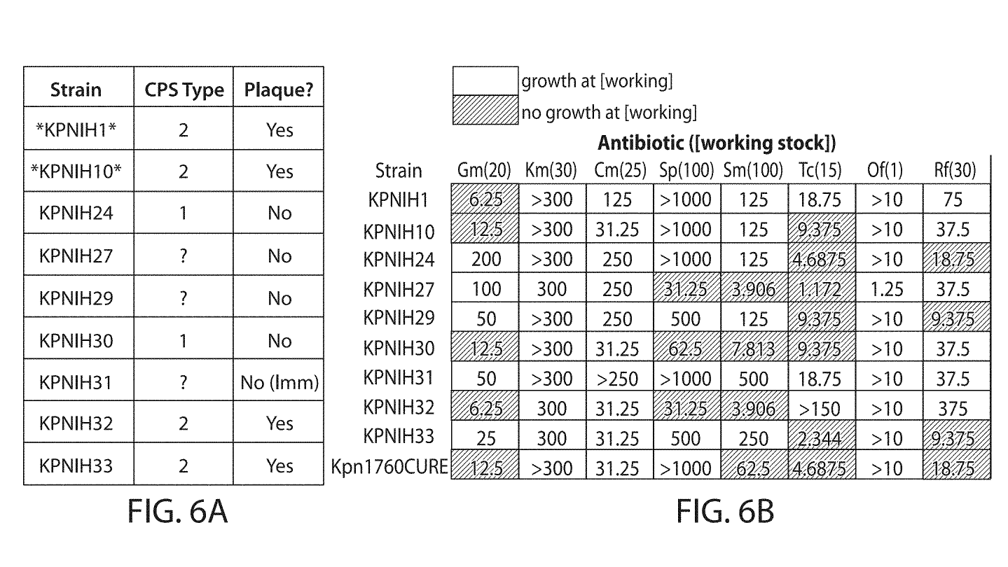

Double agar spot tests were performed to compare the lytic nature of the mutant phages relative to the wild-type phages. Wild-type N15 produces hazy plaques, owing to lysogenization of some fraction of host bacteria leading to survival and immunity against subsequent infection events instead of lysis (FIG. 5 top and bottom rows). Repressor null mutant N15 phages, whether constructed via in vitro ligation or yeast based assembly, cannot lysogenize bacteria and produce clear plaques (FIG. 5 middle rows).

### Example 2

### Cloning of Mutant Phages from Klebsiella pneumoniae and Phage Particle Production

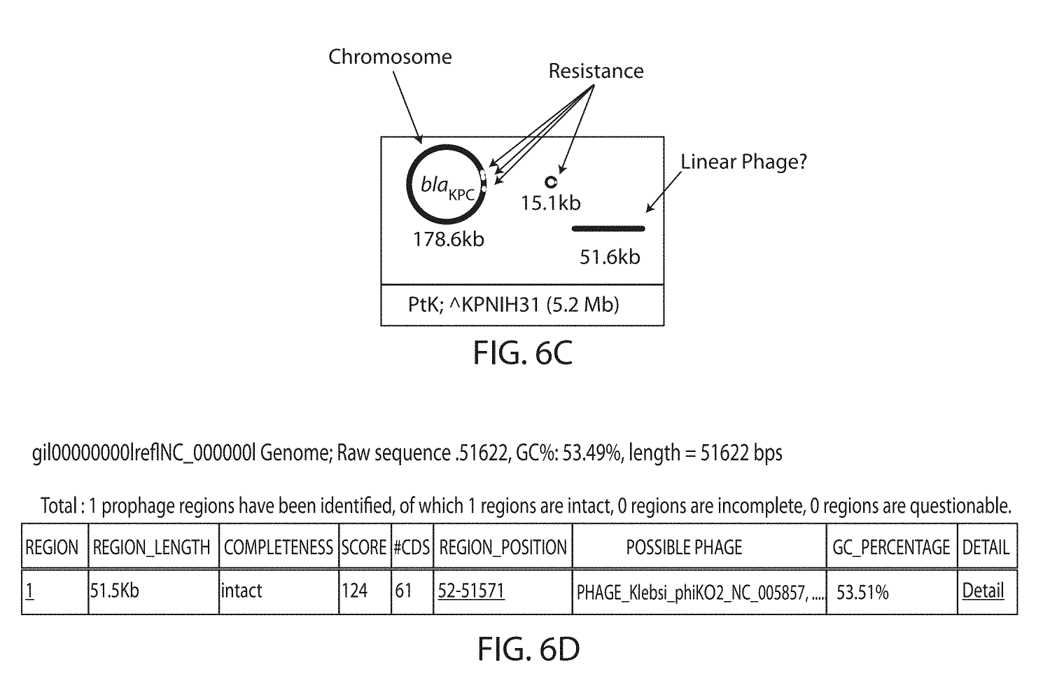

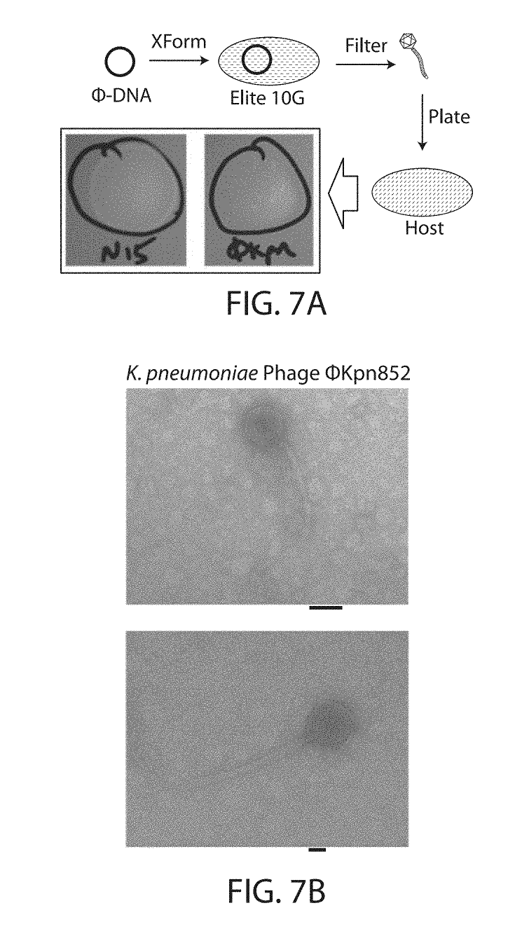

Various strains of K. pneumoniae (KPNIH) were assayed to identify the presence of and susceptibility to potential phages (FIGS. 6A-6D). Wild-type K. pneumoniae phage ΦKpn852 was isolated from strain KPNIH31, which was grown to early-log phase in LB medium. Mitomycin was then added to 1 μg/mL to induce resident prophages. Finally, crude phages were harvested, filtered, and concentrated as done with the N15 phages, which successfully produced functional phage particles (FIG. 7A). This validates the E. coli booting method for this phage. PEG-purified phage particles were visualized via transmission electron microscopy (FIG. 7B). Phage genomic DNA was purified as done with the N15 phages.

### REFERENCES

1. Bikard D., Euler C., Jiang W., Nussenzweig P. M., Goldberg G. W., Duportet X., Fischetti V. A., and Marraffini L. A., Development of sequence-specific antimicrobials based on programmable CRISPER-CAS nucleases, Nat. Biotechnol., 2014. 32(11): p. 1146-50.
2. Citorik R., Mimee M., and Lu T. K., Bacteriophage-based synthetic biology for the study of infectious diseases, Curr. Opin. Microbiol., 2014. 19: p. 59-69.
3. Conlan S., Thomas P. J., Deming C., Park M., Lau A. F., Dekker J. P., Snitkin E. S., Clark T. A., Luong K., Song Y., Tsai Y. C., Boitano M., Dayal J., Brooks S. Y., Schmidt B., Young A. C., Thomas J. W., Bouffard G. G., Blakesley R. W.; NISC Comparative Sequencing Program, Mullikin J. C., Korlach J., Henderson D. K., Frank K. M., Palmore T. N., and Segre J. A., Single-molecule sequencing to track plasmid diversity of hospital-associated carbapenemase-producing Enterobacteriaceae, Sci Transl Med., 2014. 6(254): 254ra126.
4. Hammer J. A., Jäckel C., Lanka E., Roschanski N., and Hertwig S., Binding Specificities of the Telomere Phage ΦKO2 Prophage Repressor CB and Lytic Repressor Cro, Viruses, 2016. 8(8): pii: E213.
5. Kiro R., Shitrit D., and Qimron U., Efficient engineering of a bacteriophage genome using the type I-E CRISPR-Cas system, RNA Biol., 2014. 11(1): p. 42-4.
6. Krom R. J., Krom R. J., Bhargava P., Lobritz M. A., and Collins J. J., Engineered Phagemids for Nonlytic, Targeted Antibacterial Therapies. Nano. Lett., 2015. 15(7): p. 4808-13.
7. Shin J., Jardine P., and Noireaux V., Genome replication, synthesis, and assembly of the bacteriophage T7 in a single cell-free reaction, ACS Synth. Biol., 2012. 1(9): p. 408-13.
8. Westwater C., Kasman L. M., Schofield D. A., Werner P. A., Dolan J. W., Schmidt M. G., and Norris J. S., Use of genetically engineered phage to deliver antimicrobial agents to bacteria: an alternative therapy for treatment of bacterial infections, Antimicrob. Agents Chemother., 2003. 47(4): p. 1301-07.
9. Zhou Y., Liang Y., Lynch K. H., Dennis J. J., and Wishart D. S., PHAST: a fast phage search tool, Nucleic Acids Res., 2011. 39: W347-52.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

1. A method of cloning a prophage comprising:
obtaining a prophage genome sequence,
mutating the prophage genome sequence in a sequence of the genome that decreases the function of a repressor protein, and
assembling the mutated prophage genome by either yeast assembly or in vitro assembly, optionally wherein the phage genome is isolated.

2. The method of claim 1, wherein the prophage genome is obtained by PCR, de novo synthesis, or digestion of cellular DNA.

3. The method of claim 1 or claim 2, wherein the mutated prophage genome sequence comprises a knockout of the phage repressor gene.

4. The method of any one of claims 1-3, wherein the mutated prophage genome sequence comprises at least one mutation in the sequence encoding for the phage repressor, wherein the mutation decreases the function of the repressor.

5. The method of any one of claims 1-4, wherein the mutated prophage genome sequence comprises at least one mutation in a sequence encoding at least one binding site of the phage repressor.

6. The method of any one of claims 1-5, wherein the prophage genome sequence is obtained from a phage-host cell.

7. The method of any one of claims 1-6, wherein the mutated prophage genome is further modified such that it encodes a phage that obligately kills its host cell.

8. The method of claim 7, wherein the modified genome encodes the constitutive expression of one or more toxic molecules.

9. A method of producing lytic phage particles comprising:
assembling a mutated prophage genome as in any one of claims 1-8, and
introducing the mutated prophage genome into a host cell or into an in vitro cell-free extract.

10. The method of claim 9, wherein the cell-free extract is generated from a bacterial strain.

11. The method of claim 9, wherein the cell-free extract is generated from the target strain of the lytic phage.

12. The method of any one of claims 9-11, wherein the phage particles are engineered entirely in vitro.
